(19) United States
(12) Patent Application Publication
(10) Pub. No.: US 2004/0176275 A1
(43) Pub. Date: Sep. 9, 2004

(54) Pharmaceutical composition for alleviating tissue hypoxia and methods for alleviating tissue hypoxia

(75) Inventors: Takuji Shirasawa, Tokyo (JP); Takahiko Shimizu, Tokyo (JP)

Correspondence Address: HESLIN ROTHENBERG FARLEY & MESITI PC, 5 COLUMBIA CIRCLE, ALBANY, NY, 12203, US

(73) Assignees: Tokyo Metropolitan Foundation for Research on Aging and Promotion of Welfare, Tokyo (JP); LSS CO. LTD., Tokyo (JP)

(21) Appl. No.: 10/720,431

(22) Filed: Nov. 24, 2003

(30) Foreign Application Priority Data
Mar. 6, 2003 (JP) 2003-060057

(51) Int. Cl. A61K 48/00; A61K 38/41

(52) U.S. Cl. 514/6; 514/44

(57) ABSTRACT

A pharmaceutical composition for alleviating tissue hypoxia, comprising

(1) α-globin having the Titusville mutation,

(2) a polynucleotide comprising a base sequence encoding an amino acid sequence of the α-globin having the Titusville mutation, or

(3) an expression vector comprising the polynucleotide, and a pharmaceutically acceptable carrier or diluent, is disclosed.

Further, a method for alleviating tissue hypoxia comprising administering the same to a subject, and a transgenic non-human animal having expressably a polynucleotide comprising a base sequence encoding an amino acid sequence of α-globin having the Titusville mutation, are disclosed.

### BACKGROUND OF THE INVENTION

1. Field of the Invention

The present invention relates to a pharmaceutical composition for alleviating tissue hypoxia and a method for alleviating tissue hypoxia. The pharmaceutical composition of the present invention is useful for the treatment or prevention of ischemic conditions such as respiratory failure or ischemic diseases [for example, ischemic heart diseases (for example, myocardial infarction or angina), cerebral ischemia, or obstructive arterial disorders (for example, obstructive arteriosclerosis)].

2. Description of the Related Art

In chronic respiratory failure, due to various ventilation disorders, the peripheral organs are exposed to chronic hypoxia. As such ischemic conditions (that is, diseases accompanied by tissue hypoxia), in addition to respiratory failure, various ischemic diseases such as ischemic heart diseases (for example, myocardial infarction or angina), cerebral ischemia, or obstructive arterial disorders (for example, obstructive arteriosclerosis) or the like may be mentioned. As one method of an alleviation of tissue hypoxia in these diseases, that of taking note of the oxygen affinity of hemoglobin is known.

Hemoglobin is contained in red blood cells, and is a tetrameric protein composed of four subunits which plays an important role in the delivery of oxygen from the lungs to the peripheral tissue. More specifically, it is composed of two α-chain group (α or ζ) globins and two non-α-chain group (β, γ, δ, or ε) globins, that is, a total of four globins, to each of which one hem is bonded to form a tetramer. Over 1000 mutations of hemoglobin are known (Reference 1). For example, regarding the oxygen affinity of hemoglobin, there are known mutations exhibiting a low oxygen affinity and. mutations exhibiting a high oxygen affinity.

The oxygen affinity of hemoglobin can be determined from an oxygen dissociation curve (for example, see FIG. 2 shown in Example 2). For example, mutant hemoglobin exhibiting a low oxygen affinity shows a shift to the right (rightward shift) compared with the oxygen dissociation curve of normal hemoglobin, while mutant hemoglobin exhibiting a high oxygen affinity shows a shift to the left (leftward shift).

As an agent for alleviating tissue hypoxia by causing a reduction of the oxygen affinity of hemoglobin (that is, causing a rightward shift of the oxygen dissociation curve of hemoglobin), there is known a synthetic allosteric effector containing as an active ingredient 2-[4-[[3,5-disubstituted anilino)carbonyl]methyl]phenoxy]-2-methylproprionic acid derivative (RSR13), which has been developed as a drug for the treatment of ischemic heart diseases (Reference 2).

The inventors proposed a method of utilizing Presbyterian type mutant hemoglobin, which is one of the mutant hemoglobins exhibiting a low oxygen affinity, as an approach different from the synthetic allosteric effector (Reference 3). In Presbyterian mutation, the 108th asparagine (Asn) is replaced by lysine (Lys) in the β-globin gene. The inventors prepared knock-in mice having the Presbyterian type mutation introduced to the murine β-globin gene, to study in vivo the effect of Presbyterian type mutant hemoglobin on tissues.

According to the above Reference 3, Presbyterian type mutant hemoglobin derived from heterozygous Presbyterian mutant mice exhibits a rightward shift compared with hemoglobin derived from wild-type mice.

Further, in the tibialis anterior muscle of heterozygous Presbyterian mutant mice, an increase in the oxidative enzyme rich type IIA fibers compared with wild-type mice was observed. Further, an increase in the succinate dehydrogenase (SDH) activity, an indicator of the oxidative enzymatic activity, was also observed in both type IIA fibers and type IIB fibers. These changes in the tibialis anterior muscle show that a high oxygen metabolism (for example, enhancement of the oxidative enzymatic activity) is acquired in the peripheral tissues. Further, the O₂ consumption and CO₂ production, which are parameters of respiratory metabolism, increased in heterozygous Presbyterian mutant mice, so it was confirmed that oxygen molecules could be delivered and supplied efficiently to the peripheral tissues.

Further, a spontaneous run experiment was conducted, whereupon the Heterozygous Presbyterian mutant mice exhibited daily mean running distances of values at least 2 times those of the wild-type mice. The results of this run experiment are believed to show that because oxygen molecules can be efficiently delivered and supplied to the peripheral tissues compared with wild-type mice, the tissue hypoxia at the muscle tissue during running is alleviated and the mean running distance is thereby extended. In Presbyterian mutant mice, the percentage of the Presbyterian type hemoglobin in all hemoglobin was not more than 30%, even at the maximum.

The inventors also reported on the physiological characteristics of a human Presbyterian individual in the Reference 3. In the human Presbyterian individual, a light degree of anemia was observed, but the results of a low oxygen ventilatory response examination and carbon dioxide ventilatory response examination showed that the respiration number became about half that of a healthy person due to the low oxygen and the carbon dioxide load. These results showed that, in a human Presbyterian individual, oxygen molecules were efficiently delivered and supplied to the peripheral tissues, and it became possible to continue exercise even with a respiration number of about half that of a healthy person.

From an analysis of the Presbyterian mutant mice and human Presbyterian individual, the inventors showed that Presbyterian type mutant hemoglobin exhibiting a low oxygen affinity can efficiently deliver and supply oxygen molecules to peripheral tissues, and as a result, has an effect in alleviation of tissue hypoxia, and therefore, is effective for the treatment or prevention of ischemic conditions.

References

1. “Blood”, 1998, vol. 91, p. 2643-2644

2. “The Journal of Clinical Investigation”, 1999, vol. 103, p. 739-746

3. Masakatsu Tamaki, Takahiko Shimizu, Yoichi Suzuki, and Takuji Shirasawa, “Molecular Biological Study of Treatment of Respiratory Failure”, Ministry of Health and Welfare Specific Disease Respiratory Failure Research Group FY2001 Research Report, Respiratory Failure Research Group, 2001, p. 150-154.

### SUMMARY OF THE INVENTION

The present inventors engaged in intensive research aimed at acquiring mutant hemoglobin having a superior effect in the alleviation of tissue hypoxia, even when compared with Presbyterian mutant hemoglobin, whereupon it was newly discovered that Titusville mutant hemoglobin exhibits a superior effect of the alleviation of tissue hypoxia. More specifically, in a spontaneous run experiment, heterozygous Titusville mutant mice exhibited daily mean running distances of values at least 2.5 times those of wild-type mice (with heterozygous Presbyterian mutant mice, at least 2 times). Further, with Titusville mutant mice, the percentage of the Titusville type hemoglobin in the total hemoglobin was not more than 15% even at a maximum (with Presbyterian mutant mice, 30% or less). It was found that Titusville hemoglobin, despite its low content, exhibits a superior effect of alleviation of tissue hypoxia. The present invention is based on these findings.

The object of the present invention is to provide a pharmaceutical composition for the treatment or prevention of ischemic conditions, which exhibits a superior effect of the alleviation of tissue hypoxia.

The invention relates to a pharmaceutical composition for alleviating tissue hypoxia, comprising

(1) α-globin having the Titusville mutation,

(2) a polynucleotide comprising a base sequence encoding an amino acid sequence of the α-globin having the Titusville mutation, or

(3) an expression vector comprising the polynucleotide, and a pharmaceutically acceptable carrier or diluent.

Further, the present invention relates to a method for alleviating tissue hypoxia, comprising administering to a subject in need thereof

(1) α-globin having the Titusville mutation,

(2) a polynucleotide comprising a base sequence encoding an amino acid sequence of the α-globin having the Titusville mutation, or

(3) an expression vector comprising the polynucleotide, in an amount effective therefor.

Further, the present invention relates to a method for treating or preventing ischemic conditions, comprising administering to a subject in need thereof

(1) α-globin having the Titusville mutation,

(2) a polynucleotide comprising a base sequence encoding an amino acid sequence of the α-globin having the Titusville mutation, or

(3) an expression vector comprising the polynucleotide, in an amount effective therefor.

Further, the present invention relates to a method for enhancing an oxygen metabolism in tissues, comprising administering to a subject in need thereof

(1) α-globin having the Titusville mutation,

(2) a polynucleotide comprising a base sequence encoding an amino acid sequence of the α-globin having the Titusville mutation, or

(3) an expression vector comprising the polynucleotide, in an amount effective therefor.

Further, the present invention relates to a method for modificating of a tissue, comprising administering to a subject in need thereof

(1) α-globin having the Titusville mutation,

(2) a polynucleotide comprising a base sequence encoding an amino acid sequence of the α-globin having the Titusville mutation, or

(3) an expression vector comprising the polynucleotide, in an amount effective therefor.

Further, the present invention relates to a method for enhancing exercise capacity, comprising administering to a subject in need thereof

(1) α-globin having the Titusville mutation,

(2) a polynucleotide comprising a base sequence encoding an amino acid sequence of the α-globin having the Titusville mutation, or

(3) an expression vector comprising the polynucleotide, in an amount effective therefor.

Further, the present invention relates to a method for treating or preventing cerebrovascular dementia, comprising administering to a subject in need thereof

(1) α-globin having the Titusville mutation,

(2) a polynucleotide comprising a base sequence encoding an amino acid sequence of the α-globin having the Titusville mutation, or

(3) an expression vector comprising the polynucleotide, in an amount effective therefor.

Further, the present invention relates to an artificial blood comprising α-globin having the Titusville mutation.

Further, the present invention relates to a transgenic non-human animal having expressably a polynucleotide comprising a base sequence encoding an amino acid sequence of α-globin having the Titusville mutation.

### BRIEF DESCRIPTION OF DRAWINGS

FIG. 1 schematically shows the strategy for generation of Titusville mutant mice.

FIG. 2 shows oxygen dissociation plots of red blood cells prepared from various mutant mice and wild-type mice.

FIG. 3 shows Hill's plots of red blood cells prepared from various mutant mice and wild-type mice.

FIG. 4 shows micrographs showing the results of histochemical staining based on ATPase activity or SDH activity for Titusville mutant mice and wild-type mice.

FIG. 5 is a graph of muscle fiber distribution in the tibialis anterior muscle of Titusville mutant mice and wild-type mice.

FIG. 6 is a graph of fiber SDH activity in Titusville mutant mice and wild-type mice.

FIG. 7 is a graph of the results of a spontaneous run experiment in Titusville mutant mice and wild-type mice.

FIG. 8 is a graph of the results of a spontaneous run experiment in Presbyterian mutant mice and wild-type mice.

### DESCRIPTION OF THE PREFERRED ENBODIMENTS

[1] Pharmaceutical Composition or Method for Alleviating Tissue Hypoxia of the Present Invention

The pharmaceutical composition (particularly the pharmaceutical composition for alleviating tissue hypoxia) of the present invention contains as an active ingredient

(1) α-globin having the Titusville mutation (hereinafter referred to as “Titusville α-globin”),

(2) a polynucleotide comprising a base sequence encoding an amino acid sequence of the above Titusville α-globin (1) (hereinafter referred to as a “polynucleotide for the pharmaceutical composition”), or

(3) an expression vector comprising the above polynucleotide (2) (hereinafter referred to as an “expression vector for the pharmaceutical composition”) and, as desired, further containing a pharmaceutically or veterinarily acceptable carrier or diluent.

In the method for alleviating tissue hypoxia of the present invention, at least one of the above active ingredients alone or together with a pharmaceutically or veterinarily acceptable ordinary carrier or diluent to an animal (preferably a mammal, particularly a human) in need of an alleviation of tissue hypoxia, in an effective dosage.

The “Titusville α-globin” which can be used as an active ingredient in the pharmaceutical composition of the present invention is not particularly limited, so long as it is an α-globin having at least the Titusville mutation and capable of forming a hemoglobin exhibiting a low oxygen affinity (that is, a rightward shift) . The “Titusville mutation” means a mutation in which the 94th aspartic acid (Asp) of α-globin is replaced with asparagine (Asn). The term “α-globin” as used herein is not particularly limited, so long as it is an α-globin in which the 94th amino acid is aspartic acid and the aspartic acid is replaced with asparagine so as to enable the formation of a hemoglobin exhibiting a low oxygen affinity. For example, mammalian (for example, human, murine, rat, canine, feline, simian, porcine, bovine, sheep, goat, equine, or dolphin, preferably human or murine) α-globin may be used.

As the Titusville α-globin, for example, a mutant in which the 94th aspartic acid of human α1 globin (GenBank accession number AH002715) is replaced with asparagine, a mutant in which the 94th aspartic acid of murine α-globin (GenBank accession number V00714) is replaced with asparagine, or the like may be used.

The “polynucleotide for the pharmaceutical composition” which can be used as an active ingredient in the pharmaceutical composition of the present invention is not particularly limited, so long as it comprises a base sequence encoding the amino acid sequence of the above-mentioned Titusville α-globin. For example, a polynucleotide consisting of a base sequence encoding the amino acid sequence of Titusville α-globin may be mentioned. The term “polynucleotide” as used herein includes DNA and RNA.

The “expression vector for the pharmaceutical composition” which can be used as an active ingredient in the pharmaceutical composition of the present invention is not particularly limited, so long as it comprises the above-mentioned polynucleotide for the pharmaceutical composition, in a form by which Titusville α-globin encoded by the polynucleotide can be expressed in the administered subject. For example, an expression vector in which the polynucleotide for the pharmaceutical composition is inserted in a vector for gene therapy may be mentioned.

As the vector for gene therapy, a vector comprising various sequences (for example, a promoter, a RNA splicing site, a polyadenylated site, or a transcription termination sequence) capable of expressing the inserted gene in the administered subject, such as an adenovirus vector, an adeno-associated virus (AAV) vector, or a rentivirus vector, may be mentioned.

The method of administration of the pharmaceutical composition of the present invention is not particularly limited, so long as the Titusville α-globin can function as hemoglobin in the administered subject, and may be appropriately selected in accordance with the active ingredient.

When the pharmaceutical composition of the present invention comprises the Titusville α-globin as an active ingredient, the method of administration is not particularly limited, so long as it is a method of administration enabling an injection into the vein of the administered subject as hemoglobin comprising the Titusville α-globin. For example, it may be administered as an artificial blood. As the artificial blood, for example, there are known (1) a cross-linked or polymerized hemoglobin obtained by cross-linking hemoglobin obtained from red blood cells, or (2) a liposome-type hemoglobin obtained by encapsulating hemoglobin in liposomes [Squires, J. E. , Science, 295, 1002-1004, 2002; Japanese Unexamined Patent Publication (Kokai) No. 2001-348341]. It is possible to prepare recombinant hemoglobin containing Titusville α-globin from a suitable host, such as E. coli (Looker, D. et al., Nature, 356, 258-260, 1992) or transgenic animals (O'Donnell, J. K. et al., J. Biol. Chem., 269, 27692-27699, 1994), and to administer the pharmaceutical composition of the present invention as the artificial blood.

In the pharmaceutical composition of the present invention comprising the Titusville α-globin as an active ingredient, the Titusville α-globin is preferably comprised as hemoglobin containing the Titusville α-globin. In this case, all α-chains in the hemoglobin can be the Titusville α-globin, or alternatively, a part of the α-chains can be the Titusville α-globin and the remaining α-chains can be non-Titusville α-globin (for example, a wild-type α-globin or a mutant α-globin other than the Titusville α-globin).

As shown in Examples, even with Titusville mutant mice in which the percentage of the Titusville type hemoglobin in the total hemoglobin is not more than 15% even at a maximum [see Example 2 (1)], a daily mean running distance of a value of at least 2.5 times that of wild-type mice is exhibited [see Example 2 (5)]. Therefore, with the pharmaceutical composition of the present invention comprising the Titusville α-globin as hemoglobin, it is possible to sufficiently alleviate tissue hypoxia even if only part of the α-chain in the hemoglobin is the Titusville α-globin.

When the pharmaceutical composition of the present invention comprises the polynucleotide for the pharmaceutical composition or the expression vector for the pharmaceutical composition as an active ingredient, as the method of administration, for example, the various methods in gene therapy can be used. As such a method, for example, (1) a method based on autologous transplantation or (2) a method using embryonic stem cells (ES cells) may be mentioned.

In the method based on autologous transplantation, it is possible to express hemoglobin containing the Titusville α-globin in an administered subject, for example, by harvesting bone marrow cells from the administered subject, introducing the polynucleotide or the expression vector for the pharmaceutical composition into hematopoietic stem cells in the bone marrow cells, then optionally amplifying ex vivo the gene-introduced cells, and finally returning them to the administered subject.

In the method using embryonic stem cells, it is possible to express hemoglobin containing the Titusville α-globin in an administered subject, for example, by introducing the polynucleotide or the expression vector for the pharmaceutical composition into embryonic stem cells, then causing the gene-introduced cells to differentiate into hematopoietic stem cells or hematoblasts, and finally return them to the administered subject.

For example, Pawliuk, R. et al., Science, 294, 2368-2371, 2001 discloses that sickle cell disease (SCD) accompanied by a formation of abnormal hemoglobin (HbS) derived from one base mutation of the human β^{A} globin gene is alleviated by gene therapy in SCD model mice. More specifically, an expression vector for gene therapy was constructed by designing a β^{A} globin mutation gene capable of suppressing polymerization of abnormal hemoglobin HbS, and then incorporating the gene into a rentivirus vector. By introducing the expression vector by a viral infection into hematopoietic stem cells obtained from murine bone marrow, and then returning the cells to mice, it was possible to express mutant globin in the red blood cell line over a long period of 10 months.

The dosage when using the pharmaceutical composition of the present invention can be appropriately determined in accordance with, for example, the type of disease, the age, gender, body weight, or degree of condition of the patient, the type of active ingredient, or the method of administration.

The Titusville α-globin per se as the active ingredient of the pharmaceutical composition of the present invention, or the Titusville α-globin encoded by the polynucleotide or the expression vector for the pharmaceutical composition as the active ingredient of the pharmaceutical composition of the present invention functions as hemoglobin containing the Titusville α-globin in a body of the administered subject, whereby it is possible to enhance the ability to supply oxygen from the lungs to the tissues (for example, muscles, heart, nerves, or skin). That is, it is possible to efficiently deliver and supply oxygen to the tissues, so, for example, even if the tissues fall into a hypoxia state, it is possible to alleviate the tissue hypoxia. This is useful for the treatment or prevention of ischemic conditions, for example, respiratory failure or ischemic diseases. The ischemic diseases include, for example, ischemic heart diseases (for example, myocardial infarction or angina), cerebral ischemia, or obstructive arterial disorders (for example, obstructive arterial sclerosis).

Further, the pharmaceutical composition of the present invention functions as hemoglobin containing the Titusville α-globin in a body of the administered subject, so for example exhibits the effects of

(1) enhancement of the oxygen metabolism in tissues such as muscles, heart, nerves, or skin (for example, enhancement of oxidative enzymatic activity);

(2) modification of tissues such as muscles, heart, nerves, or skin (for example, modification to muscle suitable for aerobic exercises); or

(3) enhancement of exercise capacity [for example, enhancement of running capacity (for example, extension of running distance or enhancement of tolerance to running load)].

Therefore, the pharmaceutical composition of the present invention can be used as an agent for enhancing oxygen metabolism in tissues, an agent for modifying tissues, or an agent for enhancing exercise capacity. Further, since the pharmaceutical composition of the present invention exhibits these effects, it is useful for the treatment or prevention of, for example, cerebrovascular dementia.

[2] Transgenic Non-Human Animal of the Present Invention

The transgenic non-human animal of the present invention is not particularly limited, so long as it expressably comprises a polynucleotide comprising a base sequence encoding the amino acid sequence of the Titusville α-globin. The transgenic non-human animal can be prepared by a known method (for example, Shirasawa, T. et al., J. Biol. Chem., 278, 5035-5043, 2003).

As the non-human animal, for example, a mammal other than human beings (for example, mouse, rat, dog, cat, monkey, pig, cattle, sheep, goat, horse, or dolphin), a bird (for example, chicken or quail), an amphibian (for example, frog), or reptile may be mentioned.

The transgenic non-human animal of the present invention has hemoglobin containing the Titusville α-globin, and thus can efficiently deliver and supply oxygen molecules to the peripheral tissues and exhibit a tolerance to tissue hypoxia compared with wild-type non-human animals without the Titusville α-globin. Therefore, the transgenic non-human animal of the present invention is suitable for an evaluation of agents for treating various diseases, in particular ischemic conditions, or candidate compounds therefor. For example, to evaluate agents for treating ischemic conditions or candidate compounds therefor, that is, to evaluate the action in alleviating tissue hypoxia, it is necessary to reduce a non-human animal to tissue hypoxia. In this case, with wild-type non-human animals, there is a high risk of their falling into a state where maintenance of life itself is difficult, but with the transgenic non-human animal of the present invention, tolerance is exhibited against tissue hypoxia, and thus the evaluation can be conducted.

The mechanism by which the Titusville hemoglobin exhibits a low oxygen affinity differs from the mechanism by which the Presbyterian hemoglobin exhibits a low oxygen affinity (Shirasawa, T. et al., J. Biol. Chem., 278, 5035-5043, 2003). With the Presbyterian hemoglobin, the Cl⁻ is surrounded in the hemoglobin central cavity for stabilization in the deoxygenated state by the mutation of the β108 Asn oriented toward the center of the globin tetramer to Lys. On the other hand, with the Titusville hemoglobin, it is stabilized in the deoxygenated state by the mutation of the α94 Asp positioned at the interface of the α1β2 subunits to Asn.

### EXAMPLES

The present invention now will be further illustrated by, but is by no means limited to, the following Examples.

### Example 1

### Preparation of Mutant Mice Expressing Titusville Hemoglobin

In this example, knock-in mice in which the 94th aspartic acid (Asp) in the hemoglobin α1 globin gene was replaced with asparagine (Asn) so as to introduce the Titusville mutation into the α1 globin gene were prepared in accordance with the following procedure.

An outline of the strategy is shown in FIG. 1. In FIG. 1, the symbols “α1”, “ζ”, “neo^{r}”, and “E” indicate the α1 globin gene, the ζ globin gene, the neomycin resistance gene, and the restriction enzyme EcoRI recognition site, respectively. The symbol “D94N” and the symbol “*” thereunder indicate the introduction of a mutation replacing the 94th aspartic acid (D) with asparagine (N) at the position shown by “*”.

The 372 bp of 5′ flanking sequence in the murine α1 globin gene (1st to 372nd nucleotides; GenBank accession number V00714) was used as a probe to screen the 129 Λ-phage (ΛFIXII; Strategene) libraries of the murine genome, to obtain two overlapping clones covering all exons of the gene. The 1.0 kb fragment comprising all exons of the α1 globin was amplified, then a commercially available mutagenesis kit (pALTER system; Promega) was used to modify the codon GAT corresponding to the 94th Asp to the codon AAT corresponding to Asn. A construct comprising the 5′ homologous fragment (6.8 kb), the mutated α1 globin gene (1.0 kb), the neomycin resistance gene, and 3′ homologous fragment (2.2 kb) arranged in that order was constructed in accordance with the procedure described in Shirasawa, T. et al., J. Biol. Chem., 278, 5035-5043, 2003 and cloned into the vector p-MC1DT-A(B) (Oriental Yeast).

The obtained vector was linearized by a restriction enzyme and used for electroporation of ES cells. Each genomic DNA of 240 G418-resistant clones was digested by restriction enzyme EcoRI. Clones in which the desired homologous recombination occurred were screened by Southern blot analysis using the probe shown in FIG. 1 (800 bp). The selected ES clones were used to prepare chimeric mice by the aggregation method (Hum. Reprod., 8, 2180-2184, 1993). The chimeric mice were cross-bred with C57BL/6CrSIc mice (SLC Japan), and germline transmission was confirmed by PCR amplification using the primers p1 and p2 shown in FIG. 1.

As a comparison, knock-in mice in which the 108th asparagine (Asn) in the hemoglobin β-globin gene is replaced with lysine (Lys), to introduce the Presbyterian mutation into the β-globin gene, were prepared in accordance with the procedure described in Biochem. Biophys. Res. Commun., 295, 869-876, 2002.

### Example 2

### Analysis of Mutant Mice

(1) Hemoglobin Composition and Complete Blood Cell Count

Each hemoglobin was prepared from peripheral blood of Titusville mutant mice and Presbyterian mutant mice and analyzed by reversed phase HPLC. In this connection, the preparation and analysis were performed in accordance with the procedures described in Shirasawa, T. et al., J. Biol. Chem., 278, 5035-5043, 2003. With the homozygous Titusville mutant mice, the percentage of the Titusville α-globin in all α-globin in all hemoglobin was not more than 15% even at a maximum, while with homozygous Presbyterian mutant mice, the percentage of the Presbyterian type β-globin in all β-globin in all hemoglobin was not more than 30% even at the maximum.

Further, the results of the complete blood cell count in Titusville mutant mice and wild-type mice are shown in Table 1. The data shown in Table 1 are “means±SEM”. As apparent from Table 1, with Titusville mutant mice, no abnormalities were observed.

TABLE 1

| | | Titusville |
|---|---|---|
| | Wild-type mice | mutant mice |
| RBC (×10⁶/ml) | 9.67 ± 0.35 | 9.99 ± 0.44 |
| Hb (g/dl) | 15.2 ± 0.6 | 14.6 ± 0.4 |
| Ht (%) | 56.9 ± 2.1 | 55.1 ± 1.9 |
| MCV (fl) | 59.0 ± 0.7 | 55.2 ± 0.8 |
| MCH (pg) | 16.0 ± 0.0 | 14.7 ± 0.5 |
| MCHC (g/dl) | 27.0 ± 0.0 | 26.5 ± 0.5 |
| WBC (×10³/ml) | 3.36 ± 0.18 | 2.63 ± 0.42 |
| Ret (%) | 2.2 ± 0.1 | 2.8 ± 0.7 |

(2) Analysis of Oxygen Dissociation Characteristics of Red Blood Cells

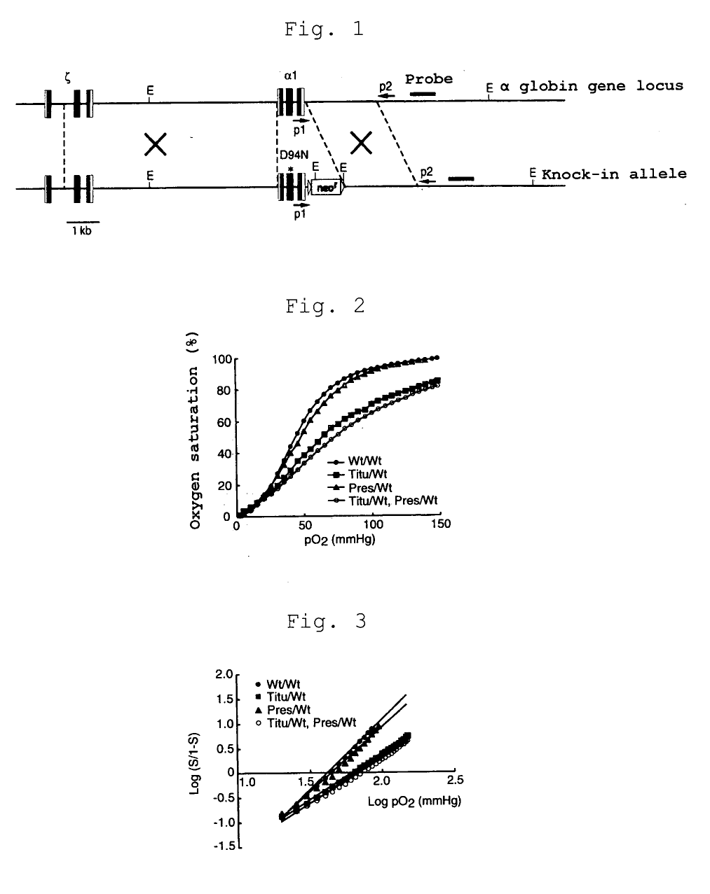

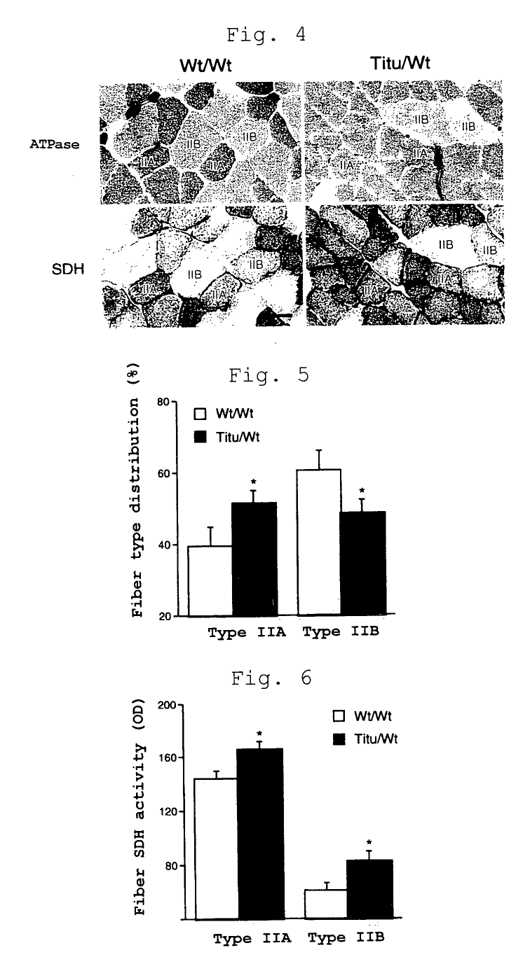

Red blood cells prepared from Titusville mutant mice (Titu/Wt), Presbyterian mutant mice (Pres/Wt), Titusville and Presbyterian double mutant mice (TiTu/Wt, Pres/Wt), and wild-type mice (Wt/Wt) were used to analyze the oxygen dissociation characteristics. The oxygen dissociation curves of red blood cells are shown in FIG. 2, and the Hill's plots of the red blood cells are shown in FIG. 3. The symbols “pO₂” in FIG. 2 and FIG. 3 mean oxygen partial pressure. In this connection, the analysis was performed using a Hemox analyzer (TCS Products) at 37° C.

In the oxygen dissociation curves shown in FIG. 2, Presbyterian hemoglobin (P₅₀=43.5 mmHg) exhibited a rightward shift compared with wild-type hemoglobin (P₅₀=47.0 mmHg). Further, Titusville hemoglobin (P₅₀=66.0 mmHg) and Titusville and Presbyterian hemoglobin (P₅₀=72.0 mmHg) exhibited further remarkable rightward shifts.

Regarding the Hill coefficient, with Presbyterian hemoglobin, no remarkable difference compared with wild-type hemoglobin was observed, while with Titusville hemoglobin and Titusville and Presbyterian hemoglobin, the Hill coefficients dropped. The drop in the Hill coefficient in the Titusville type hemoglobin suggests that a change is caused in the three-dimensional characteristics as an oxygen-binding hemoglobin tetramer.

(3) Blood Gas Analysis and Metabolism Analysis

To study the effects of mutant type hemoglobin on the acid-base balance, the pH of arterial blood, the CO₂ partial pressure in arterial blood (PaCO₂), and the O₂ partial pressure in arterial blood (PaO₂) in Titusville mutant mice, Presbyterian mutant mice, and wild-type mice were measured. The measurements were conducted in accordance with the procedures described in Shirasawa, T. et al., J. Biol. Chem., 278, 5035-5043, 2003, using a blood gas analyzer (OPTI CCA, AVL Scientific Corporation) at 37° C. The results for Titusville mutant mice and wild-type mice are shown in Table 2. The data shown in Table 2 are “means±SEM”.

With Titusville mutant mice, both in room air and under hypoxia conditions, normal pH, PaCO₂, and PaO₂ were exhibited in the same way as wild-type mice. On the other hand, with Presbyterian mutant mice, both in room air and under hypoxia conditions, a drop in pH and a rise in PaCO₂ were observed (data not shown).

TABLE 2

| | | Titusville |
|---|---|---|
| | Wild-type mice | mutant mice |
| pH | | |
| Room air | 7.42 ± 0.01 | 7.42 ± 0.03 |
| Hypoxia | 7.45 ± 0.03 | 7.46 ± 0.03 |
| PaCO₂ (mmHg) | | |
| Room air | 44.0 ± 0.9 | 41.9 ± 4.4 |
| Hypoxia | 39.6 ± 6.6 | 37.5 ± 5.6 |
| PaO₂ (mmHg) | | |
| Room air | 85.7 ± 4.2 | 84.8 ± 6.8 |
| Hypoxia | 56.2 ± 9.0 | 58.7 ± 11.0 |

Next, the O₂ consumption, CO₂ production, and respiratory exchange ratio were measured as metabolism parameters in Titusville mutant mice, Presbyterian mutant mice, and wild-type mice. The measurements were conducted in accordance with the procedures described in Shirasawa, T. et al., J. Biol. Chem., 278, 5035-5043, 2003. The results for Titusville mutant mice and wild-type mice are shown in Table 3. The data shown in Table 3 are “means±SEM”. The letters “a” and “b” show that significant differences are observed between Titusville mutant mice and wild-type mice (a: p<0.05, b: p<0.01; according to unpaired Student's test).

In both mutant mice, and with both in room air and under hypoxia conditions, more O₂ was consumed and more CO₂ was produced compared with wild-type mice.

TABLE 3

| | Wild-type | Titusville |
|---|---|---|
| | mice | mutant mice |
| CO₂ production (ml/min/kg) | | |
| Room air | 26.0 ± 1.2 | 35.1 ± 1.0^{b} |
| Hypoxia | 15.8 ± 0.8 | 20.2 ± 1.6^{a} |
| O₂ consumption (ml/min/kg) | | |
| Room air | 34.7 ± 2.0 | 42.2 ± 2.2^{a} |
| Hypoxia | 24.7 ± 1.2 | 29.8 ± 2.3^{a} |
| Respiratory exchange ratio | | |
| Room air | 0.75 ± 0.02 | 0.84 ± 0.03^{a} |
| Hypoxia | 0.64 ± 0.01 | 0.68 ± 0.01 |

(4) Histochemical and Enzymological Analyses of Muscles

To study the muscle fiber distribution of tibialis anterior muscle of Titusville mutant mice, Presbyterian mutant mice, and wild-type mice, histochemical staining was performed based on the ATPase activity. The staining was performed in accordance with the procedure described in Shirasawa, T. et al., J. Biol. Chem., 278, 5035-5043, 2003.

The results of histochemical staining at deep regions of the tibialis anterior muscle for Titusville mutant mice (Titu/Wt) and wild-type mice (Wt/Wt) are shown at the top part of FIG. 4. The muscle fiber distribution calculated based on the staining is shown in FIG. 5. The symbols “IIA” and “IIB” in FIG. 4 mean type IIA fibers and type IIB fibers. The scale bar at the bottom right corner of the bottom left side (Wt/Wt, SDH) shows the length in 50 μm. The data shown in FIG. 5 are “means±standard error” (n=5). The symbol “*” shown in FIG. 5 indicates that a significant difference (p<0.001 according to unpaired Student's test) is observed between Titusville mutant mice and wild-type mice.

In both Titusville mutant mice and Presbyterian mutant mice, no hypertrophy or atrophy of the fibers at the deep regions (side near bone), middle regions, and superficial regions (side near surface of muscle) of the tibialis anterior muscle was observed (data not shown). However, as shown in FIG. 4, in Titusville mutant mice, the percentage of type IIA fibers became higher and the percentage of type IIB fibers dropped compared with wild-type mice. Further, a similar trend was also observed in Presbyterian mutant mice. Specifically, the percentages of type IIA fibers and type IIB fibers were 39.5% and 60.5% in wild-type mice, 51.4% and 48.6% in Titusville mutant mice, and 49.8% and 50.2% in Presbyterian mutant mice, respectively. In this connection, type IIA fibers are richer in oxidative enzymes compared with type IIB fibers.

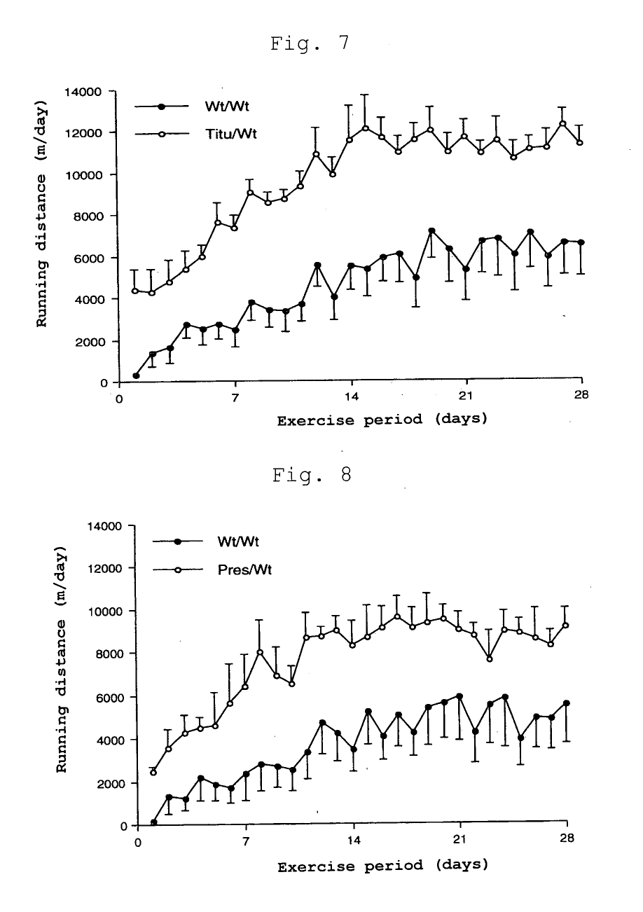

Next, the succinate dehydrogenase (SDH) activity, an indicator of the oxidative enzymes of murine tibialis anterior muscle, was measured. The histochemical staining based on the SDH activity was performed in accordance with the procedure described in Shirasawa, T. et al., J. Biol. Chem., 278, 5035-5043. The results of the histochemical staining for Titusville mutant mice and wild-type mice are shown at the bottom of FIG. 4, and the results of SDH activity in type IIA fibers and IIB type fibers calculated based on the staining are shown in FIG. 6. The data shown in FIG. 6 are “means±standard error” (n=5). The symbol “*” shown in FIG. 6 indicates that a significant difference is observed between the Titusville mutant mice and wild-type mice (p<0.001 according to unpaired Student's test).

With Titusville mutant mice, not only did the SDH activity increase in the type IIA fibers originally rich in oxidative enzymatic activity, but also an increase in the SDH activity was observed in type IIB fibers. Further, a similar trend was also observed in Presbyterian mutant mice. The change in the tibialis anterior muscle in Titusville mutant mice and Presbyterian mutant mice shows that a high oxygen metabolism is acquired at the peripheral tissues.

(5) Spontaneous Run Experiment

A spontaneous run experiment was conducted for the purpose of studying the delivery and supply capacity of oxygen to peripheral tissues in Titusville mutant mice, Presbyterian mutant mice, and wild-type mice. The daily mean running distances were compared. The spontaneous run experiment was performed by placing a running wheel (width=5 cm, diameter=25.5 cm) in a cage (20 cm×30 cm×12 cm) and following the procedure described in Shirasawa, T. et al., J. Biol. Chem., 278, 5035-5043, 2003.

The results for Titusville mutant mice (Titu/Wt) and wild-type mice (Wt/Wt) are shown in FIG. 7, while the results for Presbyterian mutant mice (Pres/Wt) and wild-type mice (Wt/Wt) are shown in FIG. 8. The data shown in FIG. 7 and FIG. 8 are “means±standard error” (n=5).

The daily mean running distance of Titusville mutant mice was a value at least about 2.5 times that of wild-type mice (with Presbyterian mutant mice, at least about 2 times that of wild-type mice). Further, as shown in the above Example 2(1), in Titusville mutant mice, the percentage of Titusville type hemoglobin in the total hemoglobin was not more than 15% even at the maximum (as much as 30% in Presbyterian mutant mice). It was confirmed that Titusville type hemoglobin, despite a low content, exhibits an oxygen delivery and supply capacity superior to that of the comparative Presbyterian type hemoglobin. This effect shows that Titusville type hemoglobin has an effect of an alleviation of tissue hypoxia superior compared with the comparative Presbyterian type hemoglobin.

The pharmaceutical composition of the present invention uses Titusville type hemoglobin exhibiting an effect superior to Presbyterian type hemoglobin, and therefore, exhibits a superior effect in the alleviation of tissue hypoxia and is useful for the treatment or prevention of ischemic conditions.

As above, the present invention was explained with reference to particular embodiments, but modifications and improvements obvious to those skilled in the art are included in the scope of the present invention.

1. A pharmaceutical composition for alleviating tissue hypoxia, comprising
(1) α-globin having the Titusville mutation,
(2) a polynucleotide comprising a base sequence encoding an amino acid sequence of said α-globin having the Titusville mutation, or
(3) an expression vector comprising said polynucleotide, and a pharmaceutically acceptable carrier or diluent.

2. A method for alleviating tissue hypoxia, comprising administering to a subject in need thereof
(1) α-globin having the Titusville mutation,
(2) a polynucleotide comprising a base sequence encoding an amino acid sequence of said α-globin having the Titusville mutation, or
(3) an expression vector comprising said polynucleotide, in an amount effective therefor.

3. A method for treating or preventing ischemic conditions, comprising administering to a subject in need thereof
(1) α-globin having the Titusville mutation,
(2) a polynucleotide comprising a base sequence encoding an amino acid sequence of said α-globin having the Titusville mutation, or
(3) an expression vector comprising said polynucleotide, in an amount effective therefor.

4. The method according to claim 3, wherein the ischemic conditions are respiratory failure, ischemic diseases, ischemic heart diseases, myocardial infarction, angina, cerebral ischemia, obstructive arterial disorders, or obstructive arteriosclerosis.

5. A method for enhancing an oxygen metabolism in tissues, comprising administering to a subject in need thereof
(1) α-globin having the Titusville mutation,
(2) a polynucleotide comprising a base sequence encoding an amino acid sequence of said α-globin having the Titusville mutation, or
(3) an expression vector comprising said polynucleotide, in an amount effective therefor.

6. The method according to claim 5, wherein the oxygen metabolism is an oxidative enzymatic activity.

7. A method for modificating of a tissue, comprising administering to a subject in need thereof
(1) α-globin having the Titusville mutation,
(2) a polynucleotide comprising a base sequence encoding an amino acid sequence of said α-globin having the Titusville mutation, or
(3) an expression vector comprising said polynucleotide, in an amount effective therefor.

8. The method according to claim 7, wherein the tissue is muscles, heart, nerves, or skin.

9. A method for enhancing exercise capacity, comprising administering to a subject in need thereof
(1) α-globin having the Titusville mutation,
(2) a polynucleotide comprising a base sequence encoding an amino acid sequence of said α-globin having the Titusville mutation, or
(3) an expression vector comprising said polynucleotide, in an amount effective therefor.

10. The method accoridng to claim 9, wherein the exercise capacity is running capacity.

11. A method for treating or preventing cerebrovascular dementia, comprising administering to a subject in need thereof
(1) α-globin having the Titusville mutation,
(2) a polynucleotide comprising a base sequence encoding an amino acid sequence of said α-globin having the Titusville mutation, or
(3) an expression vector comprising said polynucleotide, in an amount effective therefor.

12. An artificial blood comprising α-globin having the Titusville mutation.

13. A transgenic non-human animal having expressably a polynucleotide comprising a base sequence encoding an amino acid sequence of α-globin having the Titusville mutation.
